(19) United States
(12) Patent Application Publication
(10) Pub. No.: US 2026/0053103 A1
(43) Pub. Date: Feb. 26, 2026

(54) LETTUCE PLANT

(71) Applicant: TAKII & Co., Ltd, Kyoto (JP)

(72) Inventors: Ryohei Hirose, Kyoto (JP); Satoru Ishida, Kyoto (JP)

(73) Assignee: TAKII & Co., Ltd, Kyoto (JP)

(21) Appl. No.: 19/307,863

(22) Filed: Aug. 22, 2025

(30) Foreign Application Priority Data
Aug. 26, 2024 (JP) 2024-144242

(51) Int. Cl. A01H 6/14; A01H 5/10

(52) U.S. Cl. CPC A01H 6/1472; A01H 5/10

(57) ABSTRACT

The present invention provides a novel lettuce variety. The present invention provides a seed, plant, and tissue culture of a lettuce plant identified by Accession No. FERM BP-22497 and a method for producing a lettuce plant produced by crossing the lettuce plant identified by Accession No. FERM BP-22497 with any other lettuce plant.

### BACKGROUND OF THE INVENTION

### 1. Field of the Invention

The present invention relates to a lettuce plant.

### 2. Description of Related Art

Lettuce plants thrive in cool climate. Recent years have seen poor growth and diseases such as Fusarium wilt of lettuce plants, which are presumably caused by continuous cropping and high temperature and high humidity under unusual weather conditions. Under these circumstances, there is demand among producers of lettuce plants for lettuce varieties capable of being produced stably.

The following non-patent literatures are examples of related art: M. FUJINAGA, “Studies on physiological races and phylogenetic analysis of lettuce root rot pathogen, Fusarium oxysporum f. sp. Lactucae”, Journal of General Plant Pathology volume 71, page 457 (2005); and T. SHIMIZU et al., “Development Trend of Diseases on Lettuce (Lactuca sativa) of Organic Cultivation in Highlands of Nagano Prefecture and Their Control”, Annual report of the Kanto-Tosan Plant Protection Society 64:2017.12, pp. 41-46.

### SUMMARY OF THE INVENTION

In light of the foregoing, it is an object of the present invention to provide a novel lettuce plant.

In order to achieve the above object, the present invention provides a lettuce plant and seed designated as TEXLE213131. A representative sample of which is deposited under Accession No. FERM BP-22497.

The present invention also provides a lettuce plant grown from a seed identified by TEXLE213131 or Accession No. FERM BP-22497.

The present invention also provides a method for producing a lettuce plant, including the step of selfing the lettuce plant according to the present invention.

The present invention also provides a method for producing a lettuce plant, including the step of crossing the lettuce plant of the present invention with another lettuce plant.

The present invention can provide a novel lettuce plant.

### BRIEF DESCRIPTION OF DRAWINGS

FIGS. 1A and 1B show schematic diagrams illustrating examples of overlapping of upper part of leaves of a lettuce plant and the number of divisions of a leaf of a lettuce plant, respectively.

FIG. 2 shows schematic diagrams illustrating examples of the shape of a leaf of a lettuce plant.

FIGS. 3A to 3C show schematic diagrams illustrating examples of the depth of incisions of the margin of a leaf, the head size, and the shape in a longitudinal section of a head of a lettuce plant, respectively.

### DETAILED DESCRIPTION OF THE INVENTION

### <Lettuce Plant>

The lettuce plant of the present invention includes a lettuce plant designated as TEXLE213131 and identified by (deposited under) Accession No. FERM BP-22497 (hereinafter also referred to as “deposited line”) or a progeny line thereof. The lettuce plant of the present invention is characterized in that it includes a lettuce plant designated as TEXLE213131 and identified by Accession No. FERM BP-22497 or a progeny line thereof, and may include new mutation, a new transgene, and/or a new single-locus conversion. There is no particular limitation on other configurations and conditions.

The term “lettuce plant” as used herein refers to a plant that is classified under the species of L. sativa L. in the genus Lactuca in the family Asteraceae and whose scientific name is Lactuca sativa and Japanese name is chisha (chigusa, chisa). The lettuce plant may be a hybrid with a related species or a wild species, for example.

The term “lettuce plant for cultivation”, “lettuce variety for cultivation”, or “lettuce for cultivation” as used herein refers to a lettuce plant that is cultivated by humans and is superior in terms of cultivation or a variety, breeding line, or cultivar thereof. The “lettuce plant for cultivation”, “lettuce variety for cultivation”, or “lettuce for cultivation” may be a hybrid thereof or a hybrid with another lettuce species.

The term “plant” as used herein refers to a plant individual representing the whole plant.

The term “a part of a plant” as used herein refers to a part of a plant individual. Examples of “a part of a plant” include plant cells, plant protoplasts, plant cell cultures or tissue cultures from which a plant body can be regenerated, plant calli, plant clumps, plant cells isolated from the plant or a part of the plant, leaves, heading leaves, expanded leaves, pollen, embryos, cotyledons, hypocotyls, roots, tips of roots (root tips), anthers, pistils, flowers, ovaries, ovules, seeds, fruits, stems, and seedlings. The part of the plant individual may be an organ, tissue, a cell, or a propagule, for example, and may be any of them. Examples of the organ include petals, corollas, flowers, leaves, seeds, fruits, stems, and roots. The tissue is a part of the organ, for example. Specific examples of the part of the plant individual include microspores, flowers, flower buds, pistils, anthers, pollen, ovaries, embryos, ovules, hypocotyls, embryonic sacs, egg cells, cuttings, roots, root tips, trunks, stems, leaves, heading leaves, expanded leaves, petioles, pith of leaves, cotyledons, cells, meristematic cells, protoplasts, and seeds. The pollen may be mature pollen or immature pollen. The part of the plant individual may be derived from, for example, a plant at any growth stage, and may be derived from, for example, a pre-rooting individual, a post-rooting individual, a seedling, a cutting, or a mature individual. The part of the plant individual may be one type of organ, tissue, and/or cell, or two or more types of organs, tissue, and/or cells, for example.

The term “Fusarium wilt (lettuce Fusarium wilt)” as used herein refers to a disease caused by filamentous fungi. Examples of the pathogen of the Fusarium wilt include races such as Fusarium oxysporum f. sp. Lactucae Race 1. The other pathogen of the Fusarium wilt is reported, Race 2 and Yuma isolate isolate, for example. The term “races” refers to fungal strains that exhibit different pathogenicity, more specifically, fungal strains that exhibit different pathogenicity to varieties having different resistance genes or resistance loci. The term “Yuma isolate isolate” as used herein refers to a race of pathogen isolated from the soil of Yuma, Arizona, United States. The pathogen of Yuma isolate isolate has capable of causing lettuce Fusarium wilt in lettuce cultivars such as “Costa Rica No. 4” and “Banchu Red Fire”.

The term “resistance” as used herein also may be referred to as “tolerance” or “tolerant”, for example. The resistance means the ability to inhibit or suppress the occurrence and progression of a disease caused by the infection with the pathogen, for example. Specifically, the resistance may mean any of the following, for example: to prevent the disease from occurring; to stop the progression of the disease that has occurred already; and to suppress (also referred to as “inhibit”) the progression of the disease that has occurred already.

In the present invention, the “Fusarium wilt resistance” can be evaluated according to a method for measuring Characteristic No. 59 to be described below. In the present invention, a lettuce plant exhibiting the “Fusarium wilt resistance” need only to exhibit resistance to at least one of the races of Fusarium oxysporum f. sp. Lactucae and may exhibit resistance to two or more of the races. Preferably, the lettuce plant exhibits resistant to Fusarium oxysporum f. sp. Lactucae (Fol) Race 1 and/or Fusarium oxysporum f. sp. Lactucae (Fol) Yuma isolate isolate.

### <Deposited Line>

The lettuce plant of the present invention is designated as TEXLE213131, and a representative example thereof is a lettuce plant identified by Accession No. FERM BP-22497 or a progeny line thereof. Hereinafter, the lettuce plant identified by Accession No. FERM BP-22497 is also referred to as a lettuce line TEXLE213131. Information on the deposit of this variety is shown below.

### (TEXLE213131)

Type of deposit: International deposit
Name of depository institution: National Institute of Technology and Evaluation, International
Patent Organism Depositary; NITE-IPOD
Address: 2-5-8-120, Kazusakamatari, Kisarazu-shi, Chiba 292-0818, Japan
Accession number: FERM BP-22497
Identifying designation: TEXLE213131
Date of acceptance: May 16, 2024

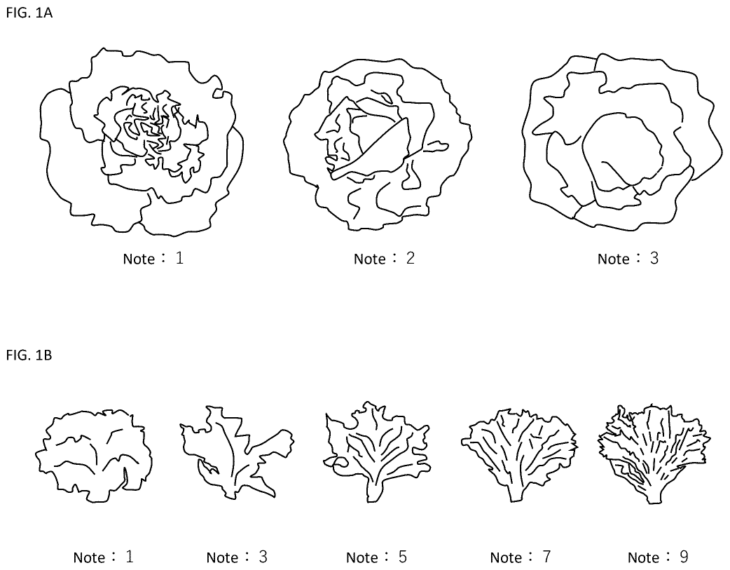

The deposited line exhibits morphological and physiological characteristics as well as resistant to Fusarium oxysporum f. sp. Lactucae (Fol) Yuma isolate as described in Table 1 below, for example. Table 1 below shows the morphological and physiological characteristics of lettuce plants cultivated at Takii Shiojiri Breeding Station in Shiojiri-shi, Nagano, Japan in 2023. The morphological and physiological characteristics shown in Table 1 were evaluated according to the Test Guidelines for Lettuce (Lactuca sativa L.) (issued in September 2022, https://www.maff.go.jp/j/shokusan/hinshu/info/kijun/1361.pdf) provided by the Ministry of Agriculture, Forestry and Fisheries of Japan (MAFF). Also, the morphological and physiological characteristics shown in Table 1 were evaluated according to criteria to be described below. For the morphological and physiological characteristics, reference can be made to FIGS. 1 to 3. Further, for the evaluation of the morphological and physiological characteristics, reference may be made to the Guidelines for the Conduct of Tests for Distinctness, Uniformity and Stability (DUS Test Guidelines) provided by the International Union for the Protection of New Varieties of Plants (UPOV). For the DUS test guidelines, reference can be made to the guidelines issued on Apr. 5, 2017 (https://www.upov.int/edocs/tgdocs/en/tg013.pdf), for example.

TABLE 1

| Characteristic | | State of |
|---|---|---|
| No. | Characteristics | TEXLE213131 |
| 1 | Seed: color | White |
| 4 | Plant: diameter | Medium |
| 5 | Plant: degree of overlapping of | Strong |
| | upper part of leaves | |
| 8 | Leaf: number of divisions | Absent or very few |
| 9 | Leaf: shape | Narrow oblate |
| 13 | Leaf: anthocyanin coloration | Absent or very weak |
| 22 | Leaf: blistering | Slightly strong |
| 24 | Leaf: undulation of margin | Medium |
| 26 | Leaf: depth of incisions of margin | Medium |
| 30 | Head: size | Medium |
| 31 | Head: shape in longitudinal section | Narrow oblate |
| 40 | Time of beginning of bolting | Slightly late |
| 59 | Resistance to Fusarium oxysporum | Highly resistant |
| | f. sp. Lactucae (Fol) Race 1 | |

### (Characteristic No. 1)

The “Seed: color” can be evaluated by visual observation. The “Seed: color” can be evaluated according to the following criteria: Note 1 (white, example variety: Olympia), Note 2 (yellow), Note 3 (brown, example varieties: Cisco and V Lettuce), and Note 4 (black, example variety: Logic).

### (Characteristic No. 4)

The “Plant: diameter” refers to a maximum diameter (cm) of a plant and can be evaluated by measurement. The “Plant: diameter” can be evaluated according to the following criteria: Note 1 (very small), Note 3 (small, example variety: Okayama Saradana), Note 5 (medium, example variety: Olympia), Note 7 (large), and Note 9 (very large).

### (Characteristic No. 5)

The “Plant: degree of overlapping of upper part of leaves” refers to the degree of overlapping of upper part of leaves (the degree of heading) of a plant and can be evaluated by visual observation. The “Plant: degree of overlapping of upper part of leaves” can be evaluated according to the following criteria: Note 1 (absent or weak, example variety: Prize Head), Note 2 (medium, example variety: Costa Rica No. 4), and Note 3 (strong, example variety: Olympia and Vanguard 75).

### (Characteristic No. 8)

The “Leaf: number of divisions” refers to the number of divisions of a leaf and can be evaluated by visual observation. The “Leaf: number of divisions” can be evaluated according to the following criteria: Note 1 (absent or very few, example variety: Olympia), Note 3 (few), Note 5 (medium), Note 7 (many), and Note 9 (very many).

### (Characteristic No. 9)

The “Leaf: shape” refers to the shape of a leaf of lettuce varieties in which the number of divisions of a leaf is absent or very few (only varieties in which the number of divisions of a leaf is absent or very few are targets of evaluation of this characteristic) and can be evaluated by visual observation. The “Leaf: shape” can be evaluated according to the following criteria: Note 1 (triangular), Note 2 (lanceolate), Note 3 (medium oblate, example variety: Olympia), Note 4 (narrow oblate), Note 5 (circular, example variety: White Boston), Note 6 (broad elliptic), Note 7 (medium elliptic, example variety: Costa Rica No. 4), Note 8 (narrow elliptic, example variety: Akakakichisya), Note 9 (linear), Note 10 (broad obtrullate), Note 11 (obovate, example variety: Prize Head), and Note 12 (oblanceolate).

### (Characteristic No. 13)

The “Leaf: anthocyanin coloration” refers to the degree of anthocyanin coloration of a leaf and can be evaluated by visual observation. The “Leaf: anthocyanin coloration” can be evaluated according to the following criteria: Note 1 (absent or very weak), Note 3 (weak), Note 5 (medium, example variety: Prize Head), Note 7 (Strong), and Note 9 (vary strong).

### (Characteristic No. 22)

The “Leaf: blistering” refers to the degree of blistering (crinkling) on a leaf surface and can be evaluated by visual observation. The “Leaf: blistering” can be evaluated according to the following criteria: Note 1 (absent or very weak, example variety: Cisco), Note 3 (weak, example variety: Olympia), Note 5 (medium, example variety: Early Impulse), Note 7 (strong, example variety: Nishina Beni), and Note 9 (very strong, example variety: Black Seeded Simpson).

### (Characteristic No. 24)

The “Leaf: undulation of margin” refers to the degree of undulation of the margin of a leaf and can be evaluated by visual observation. The “Leaf: undulation of margin” can be evaluated according to the following criteria: Note 1 (absent or very weak), Note 3 (weak), Note 5 (medium, example variety: Mikado Great 3204), Note 7 (strong, example varieties: Calmar and Grand Rapids), and Note 9 (very strong).

### (Characteristic No. 26)

The “Leaf: depth of incisions of margin” refers to the depth of incisions of the margin of a leaf and can be evaluated by visual observation. The “Leaf: depth of incisions of margin” can be evaluated according to the following criteria: Note 1 (absent or very shallow), Note 3 (shallow), Note 5 (medium, example variety: Olympia), Note 7 (deep), and Note 9 (very deep).

### (Characteristic No. 30)

The “Head: size”, which means the size of a head, refers to the head size (head diameter×head height) of varieties in which the degree of overlapping of upper part of leaves of a plant is medium or strong (only varieties in which the degree of overlapping of upper part of leaves of a plant is medium or strong are targets of evaluation of this characteristic) and can be evaluated by measurement and visual observation. The “Head: size” can be evaluated according to the following criteria: Note 1 (very small), Note 3 (small), Note 5 (medium, example variety: Olympia), Note 7 (large), and Note 9 (very large)

### (Characteristic No. 31)

The “Head: shape in longitudinal section” refers to the shape in a longitudinal section of the head of varieties in which the degree of overlapping of upper part of leaves of a plant is medium or strong (this characteristic applies only to varieties in which the degree of overlapping of upper part of leaves of a plant is medium or strong) and can be evaluated by visual observation. The “Head: shape in longitudinal section” can be evaluated according to the following criteria: Note 1 (narrow elliptic), Note 2 (broad elliptic), Note 3 (circular, example variety: Excel Head Glass), and Note 4 (medium oblate, example variety: Cisco).

### (Characteristic No. 40)

The “Time of beginning of bolting” refers to how fast bolting starts under long-day conditions (the time at which 50% of test plants start to bolt; the number of days elapsed since the sowing date) and can be evaluated by measurement and visual observation. The “time of beginning of bolting” can be evaluated according to the following criteria: Note 1 (very early, example variety: Green Leaf), Note 3 (early, example variety: Prize Head), Note 5 (medium, example varieties: Get and Falcon), Note 7 (late, example variety: Olympia), and Note 9 (very late).

### (Characteristic No. 59)

The “Resistance to Fusarium oxysporum f. sp. Lactucae (Fol) Race 1” refers to the degree of resistance to Fusarium oxysporum f. sp. Lactucae (Fol) Race 1. The “Resistance to Fusarium oxysporum f. sp. Lactucae (Fol) Race 1” can be evaluated according to the following criteria: Note 1 (susceptible, example varieties: Patriot, Banchu Red Fire, Waldmann's Green), Note 2 (moderately resistant, example variety: Salinas 88), and Note 3 (highly resistant, example variety: Costa Rica No. 4).

The resistance to Fusarium oxysporum f. sp. Lactucae (Fol) Race 1 can be examined by the following test method.
Test Method
   Pathogen: Prepare Fusarium oxysporum f. sp. Lactucae (Fol) Race 1 for the test. A susceptible variety is used for confirmation of the race.
   Confirmation of pathogenicity: Confirm the pathogenicity of the race using a susceptible variety prior to the test.
Conduct of Test
   The number of test individuals: at least 30 individuals, which should be divided for two or more replicates (in the case of a non-inoculated area, prepare at least 20 individuals, which should be divided for two or more replicates).
   Example varieties: Patriot, Banchu Red Fire, and Waldmann's Green as susceptible varieties; Salinas 88 as a moderately resistant variety; and Costa Rica No. 4 as a highly resistant variety.
   Temperature: Control the test individuals generally at 20° C. at night and in the range from 25° C. to 28° C. during the day.
   Illuminance: natural light; or illumination with at least 15000 1× and a light period of 14 hours
   Preparation for Inoculation: Inoculate the test fungal strain cultured in a nutrient medium (PSA medium or the like) into a bran medium, mix the strain well with the medium, and then culture the strain at about 25° C. for 10 to 14 days. The medium throughout which the strain has grown sufficiently is used as an inoculum.
   Inoculation Method: Add the inoculum to sterilized soil (or commercially available culture soil) with the volume ratio of the soil to the inoculum being 20:1 and mixed them together well. Fill pots or the like with this contaminated soil, and sow healthy seeds after being subjected to treatment to promote germination. At this time, avoid sowing weakly germinating seeds as much as possible, because, if withering occurs, it would be difficult to determine whether the withering is caused by the disease.
   Test Period: The disease severity is evaluated 20 to 30 days after the inoculation (sowing).
   Evaluation Criteria: The disease severity is evaluated for each individual according to the following criteria, and the disease index is calculated to make determination.
   Disease severity 0: no disease symptoms
   Disease severity 1: mild stunting, growth suppression
   Disease severity 2: severe stunting
   Disease severity 3: withering

Disease index=Σ(disease severity×the number of individuals with each disease severity)/(the total number of individuals)
Determination: Evaluation is made based on comparison with the disease indexes of the respective example varieties.
In addition to the above-described test method, it is also possible to conduct the test by a seedling dipping method. For the details of the seedling dipping method, reference can be made to, for example, the Guidelines for the Conduct of Tests presented by the UPOV (LETTUCE; TG/13/11 Rev. 2, https://www.upov.int/edocs/tgdocs/en/tg013.pdf).

In the present invention, the deposited line may exhibit resistant to Fusarium oxysporum f. sp. Lactucae (Fol) Yuma isolate in addition to morphological and physiological characteristics as described in Table 1. The “Resistant to Fusarium oxysporum f. sp. Lactucae (Fol) Yuma isolate” can be evaluated according to the following criteria: Resistant; example variety: Meridian, and Susceptible; example varieties: Patriot.

The resistant to Fusarium oxysporum f. sp. Lactucae (Fol) Yuma isolate can be examined by the described test method above as the method for the resistance to Fusarium oxysporum f. sp. Lactucae (Fol) Race 1. The pathogen isolated from the soil in Yuma, Arizona, in the United States should be used for the test for the resistant to Fusarium oxysporum f. sp. Lactucae (Fol) Yuma isolate.

In the present invention, a plant having “essentially all physiological and morphological characteristics of the deposited line” means a plant that has the main characteristics of the deposited line from which the plant of interest is derived when it is grown in the same environment. The main characteristics are the following characteristics (1) to (14), i.e., Characteristic Nos. 40, 59, 1, 4, 5, 8, 9, 13, 22, 24, 26, 30, and 31 shown in Table 1 above as well as resistant to Fusarium oxysporum f. sp. Lactucae (Fol) Yuma isolate. The main characteristics are preferably Characteristic Nos. 40, and 59 in Table 1 above as well as resistant to Fusarium oxysporum f. sp. Lactucae (Fol) Yuma isolate, i.e., the following characteristics (1), (2), and (14). The plant having “essentially all physiological and morphological characteristics of the deposited line” may be, for example, a plant having the same characteristic(s) as the deposited line, except for 14 or less, 13 or less, 12 or less, 11 or less, 10 or less, 9 or less, 8 or less, 7 or less, 6 or less, 5 or less, 4 or less, 3 or less, 2 or less, or 1 characteristic. In other words, the plant may be different from the deposited line in 14 or less, 13 or less, 12 or less, 11 or less, 10 or less, 9 or less, 8 or less, 7 or less, 6 or less, 5 or less, 4 or less, 3 or less, 2 or less, or 1 characteristic. The “different characteristic(s) from the deposited line” may be a main characteristic(s) of the deposited line or a characteristic(s) other than the main characteristics of the deposited line, and is preferably a characteristic(s) other than the main characteristics of the deposited line. The “different characteristic(s) from the deposited line” can be conferred by, for example, introduction of a characteristic(s) and/or introduction of a gene(s), which will be described below. In the plant having “essentially all physiological and morphological characteristics of the deposited line”, all the characteristics of Characteristic Nos. 40, 59, 1, 4, 5, 8, 9, 13, 22, 24, 26, 30, and 31 as well as resistant to Fusarium oxysporum f. sp. Lactucae (Fol) Yuma isolate may be the same as those of the deposited line.
(1) time of beginning of bolting is “slightly late”;
(2) resistance to Fusarium oxysporum f. sp. Lactucae (Fol) Race 1 is “highly resistant”;
(3) the seed color is “white”;
(4) the plant diameter is “ ”;
(5) the degree of overlapping of upper part of leaves of a plant is “strong”;
(6) the number of divisions of a leaf is “absent or very few”;
(7) the leaf shape is “narrow oblate”;
(8) anthocyanin coloration of a leaf is “absent or very weak”;
(9) leaf blistering is “slightly strong”;
(10) undulation of the margin of a leaf is “medium”;
(11) the depth of incisions of the margin of a leaf is “medium”;
(12) the size of a head is “medium”;
(13) the shape in a longitudinal section of a leaf is “medium oblate”; and
(14) resistant to Fusarium oxysporum f. sp. Lactucae (Fol) Yuma isolate is “resistant”

### <Progeny Line>

The lettuce plant of the present invention may be a progeny line of the deposited line. The progeny line may be a plant individual of the progeny line, a part of a plant individual of the progeny line, or a seed of the progeny line.

In the present invention, a “progeny line” or “progeny lettuce plant” (collectively referred to as “progeny line” hereinafter) refers to a plant obtained from a lettuce plant of the deposited line or from a progeny line thereof. In the present invention, the progeny line may be a plant obtained by crossing the deposited line with another lettuce plant or by crossing the deposited line with a wild lettuce plant. The progeny line may be directly or indirectly obtained, obtainable, or derived from the deposited line or a progeny line thereof by selfing and/or cross-pollination, or may be derived from a parental line obtained from the deposited line using a traditional breeding method such as selfing and/or cross-pollination. The progeny line may be, for example, a selfed progeny line or a first-generation hybrid F1 (hybrid first-generation line, F1 hybrid). When obtaining the progeny line, the deposited line may be used as a female parent, a male parent, or both parents.

The term “crossing” as used herein refers to the crossing of two parent lines. The crossing may be “cross-pollination” or “self-pollination”. Cross-pollination means fertilization by the union of two gametes that are derived from different plants. Self-pollination means transfer of pollen from the anthers to the stigma of the same plant. Self-pollination can also be referred to as selfing, for example. The crossing may encompass backcrossing, which is one of traditional breeding methods.

The “backcrossing” is one of traditional breeding techniques and is a method in which a breeder introduces a characteristic into a plant or a variety by repeatedly crossing a hybrid progeny line with one of the parental lines. A plant including the characteristic to be introduced may be referred to as a donor plant, for example. A plant into which the characteristic is to be introduced may be referred to as a recurrent parent, for example. The backcrossing can be performed by crossing the donor plant with the recurrent parent, and a first-generation hybrid F1 (hybrid first-generation line, F1 hybrid) can be obtained by the first crossing. Next, a progeny line having the above-described characteristic is crossed with the recurrent parent. Then, through backcrossing and/or selfing over several generations, the characteristic of the donor plant can be introduced into the recurrent parent.

In the present invention, the progeny line may be: regenerated from a cell culture or tissue culture, protoplast, or part of a plant individual, each derived from the deposited line; obtained by selfing the deposited line; or obtained by producing seeds from a plant individual of the deposited line.

The term “regenerate” as used herein refers to the development or vegetative propagation of a plant from a cell culture, tissue culture, or protoplast.

The “tissue culture” or “cell culture” may be a composition containing the same type or different types of isolated cells or may be an aggregate of cells that organize into a part of a plant. Tissue cultures of various types of tissue of lettuce plants and methods for regenerating plants from the tissue cultures are well known, and reference can be made to Reference Literatures 1 to 3 below, for example.
Reference Literature 1: Pink D. A. and Carter P. J., “Propagation of lettuce (Lactuca sativa) breeding material by tissue culture”, Ann. Appl. Biol., 1987, Vol. 110, pages 611-616
Reference Literature 2: Ampomah-Dwamena C., Conner A. J. and Fautrier A. G., “Genotypic response of lettuce cotyledons to regeneration in vitro”, Sci. Hort., 1997, Vol. 71, pages 137-145
Reference Literature 3: Takayuki Mizutani and Takayuki Tanaka, “Retasu oyobi Kinenyaseishu Lactsuca serriola no Kakei no Setsubaiyo (Nodal Culture of Flower Stalk of Lettuce, Lactuca sativa and L. serriola)”, Engeigaku Kenkyu (Horticultural Research (Japan)), 2008, Vol. 7 (1), pages 17 to 21

The progeny line may have desired characteristics. The progeny line may have “essentially all physiological and morphological characteristics of the deposited line” when it is cultivated under the same cultivation conditions, for example. Specifically, the progeny line may have a characteristic(s) in common with a corresponding deposited line (the deposited line from which the progeny line is derived). As specific examples, 1 or more, 2 or more, 3 or more, 4 or more, 5 or more, 6 or more, 7 or more, 8 or more, 9 or more, 10 or more, 11 or more, 12 or more, 13 or more, or 14 or more characteristics of the progeny line match the characteristics of the deposited line. The progeny line may be a plant having a main characteristic(s) of the deposited line. The main characteristics may be Characteristic Nos. 40, and 59 shown in Table 1 above as well as resistant to Fusarium oxysporum f. sp. Lactucae (Fol) Yuma isolate, i.e., the above-described characteristics (1), (3) and (14). Also, the main characteristics may be Characteristic Nos. 1, 4, 5, 8, 9, 13, 22, 24, 26, 30, and 31 in Table 1 above, i.e., the above-described characteristics (3) to (13). The progeny lines may be, for example, plants having the same characteristic as the deposited line, except for 14 or less, 13 or less, 12 or less, 11 or less, 10 or less, 9 or less, 8 or less, 7 or less, 6 or less, 5 or less, 4 or less, 3 or less, 2 or less, or 1 characteristic. In other words, the plant may be different from the deposited line in 14 or less, 13 or less, 12 or less, 11 or less, 10 or less, 9 or less, 8 or less, 7 or less, 6 or less, 5 or less, 4 or less, 3 or less, 2 or less, or 1 characteristic. The “different characteristic(s) from the deposited line” may be a main characteristic(s) of the deposited line or a characteristic(s) other than the main characteristics of the deposited line, and is preferably a characteristic(s) other than the main characteristics of the deposited line. The “different characteristic(s) from the deposited line” can be conferred by, for example, introduction of a characteristic(s) and/or introduction of a gene(s), which will be described below. In the progeny line, all the characteristics of Characteristic Nos. 40, 59, 1, 4, 5, 8, 9, 13, 22, 24, 26, 30, and 31 as well as resistant to Fusarium oxysporum f. sp. Lactucae (Fol) Yuma isolate may be the same as those in the deposited line. Examples of the different characteristic from the deposited line include resistance to Bremia Lactucae (B1: 16 to 37, Bl: 5US to 11US), tolerance to bacterial rot, tolerance to corky root, tolerance to lettuce mosaic virus, resistance to lettuce aphids (Nasonovia ribisnigri), resistant to pea leaf miners (Liriomyza huidobrensis), and resistance to browning at cut surfaces. Each characteristic can be introduced by, for example, crossing with a known plant having a locus associated with the characteristic.

The progeny line obtained by the “selfing”, or the lettuce plant obtained by the “regenerate” from the deposited line or a progeny line thereof have “essentially all physiological and morphological characteristics of the deposited line” when it is cultivated under the same cultivation conditions.

The progeny line may include cells containing at least one set of chromosomes derived from the deposited line. For example, at least about 10%, 20%, 30%, 40%, 50%, 55%, 60%, 65%, 70%, 80%, 85%, 90%, 95%, 96%, 97%, 98%, or 99% of allele of the progeny line may be derived from the deposited line. That is, the progeny line may have at least about 10%, 20%, 30%, 40%, 50%, 55%, 60%, 65%, 70%, 80%, 85%, 90%, 95%, 96%, 97%, 98%, or 99% genetic complement with the deposited line.

The “allele(s)” refers to one or more genes, all of which are associated with a trait or characteristic of a lettuce plant. In a diploid cell or organism, a pair of alleles of a given gene occupy the corresponding genetic loci on a pair of homologous chromosomes.

The genetic complement can be calculated by, for example, decoding a molecular marker or base sequence of the progeny line, comparing it with a molecular marker or base sequence of TEXLE213131, and calculating the concordance rate. Examples of the molecular marker include SNP (single nucleotide polymorphism) markers, amplified fragment length polymorphism (AFLP) markers, restriction fragment length polymorphism (RFLP) markers, microsatellite markers, sequence-characterized amplified region (SCAR) markers, and cleaved amplified polymorphic sequence (CAPS) markers. Methods for analyzing genomes using the molecular markers are well known and widely open to the public (e.g., Reference Literatures 4 and 5 below). The base sequence can be decoded by, for example, extracting a genome DNA from the progeny line and sequencing the genome DNA. The percentage of alleles derived from the deposited line and the percentage of genetic complement may each be estimated on the basis of the number of times of crossing, for example. In this case, the percentage can be estimated on the basis of the number of times of crossing from the deposited line. As a specific example, when the number of times of crossing from the deposited line is n, the percentage can be estimated as per {1−(½)ⁿ}×100%, for example.
Reference Literature 4: Sinchan Adhikari et al., “Application of molecular markers in plant genome analysis: a review”, The Nucleus, 2017, Volume 60, Issue 3, pp. 283-297 Reference Literature 5: Elcio P. Guimaraes et al., “MARKER-ASSISTED SELECTION Current status and future perspectives in crops, livestock, forestry and fish”, 2007, Springer, pp. 29-49.

Preferably, the percentage of alleles derived from the deposited line and the percentage of genetic complement are each an average value of the percentages determined with respect to a plurality of progeny lines, for example. The “plurality” refers to, for example, the number of individuals sufficient to enable statistical examination, and specifically refers to, for example, at least 200 individuals and preferably 200 to 1000 individuals.

The progeny line may have a polymorphism(s) of molecular markers derived from the deposited line. For example, at least about 10%, 20%, 30%, 40%, 50%, 55%, 60%, 65%, 70%, 80%, 85%, 90%, 95%, 96%, 97%, 98%, or 99% of the molecular markers of the progeny line may be derived from the deposited line. That is, at least about 10%, 20%, 30%, 40%, 50%, 55%, 60%, 65%, 70%, 80%, 85%, 90%, 95%, 96%, 97%, 98%, or 99% of the molecular markers of the progeny line may match the molecular markers of the deposited line. In the present invention, when, for example, 50%, 55%, 60%, 65%, 70%, 80%, 85%, 90%, 95%, 96%, 97%, 98%, or 99% or more of molecular markers of a lettuce plant of interest match the molecular markers of the deposited line, the lettuce plant of interest can be determined (discriminated, estimated, appraised, or assessed) as being a progeny line of the deposited line.

The progeny line and the lettuce line TEXLE213131 may have, for example, mutation or a transgene. In this case, for example, one, two, three, four or more characteristics or transgenes are introduced into the progeny line, whereby the characteristics of the progeny line are modified accordingly. The progeny line can be produced by, for example, introducing mutation or a transgene into the deposited line or a progeny line thereof. The mutation may be introduced artificially or naturally, and artificial introduction of the mutation can also be referred to as induction of the mutation. The mutation may be, for example, a chemical-induced mutation or a radiation-induced mutation. Also, the mutation may be introduced by, for example, a molecular biological procedure or a genome-editing technique (e.g., Reference Literature 6 below). The mutation or transgene may be introduced into a lettuce seed, lettuce plant, cell culture, and/or tissue culture of the deposited line. The transgene can be introduced by, for example, a method using Agrobacterium tumefaciens.
Reference Literature 6: Yanfei Mao et al., “Gene editing in plants: progress and challenges”, National Science Review, 2019, Vol. 6, pp. 421-437

Examples of the above-described one or more characteristics include resistance to Bremia Lactucae (B1: 16 to 37, Bl: 5US to 11US), tolerance to bacterial rot, tolerance to corky root, tolerance to lettuce mosaic virus, resistance to lettuce aphids (Nasonovia ribisnigri), resistant to pea leaf miners (Liriomyza huidobrensis), and resistance to browning at cut surfaces.

The transgene refers to a desired gene introduced into the genome of a plant by, for example, a genetic engineering procedure or a traditional breeding method. The transgene may include, for example, a gene introduced through single-locus conversion. The transgene may be derived from the same species or a different species, for example. The transgene may include the same base sequence as the species from which it is derived or may include a different base sequence from the species from which it is derived. In the latter case, the different base sequence can be prepared by, for example, codon optimization of the above-described same base sequence or adding a transcriptional regulator such as a promoter to the above-described same base sequence. The transgene may have translated regions and untranslated regions.

### <Haploid Plant and Doubled Haploid Plant>

The lettuce plant of the present invention may be a haploid plant and/or doubled haploid plant that is obtained, obtainable, or induced from the deposited line. The haploid plant and/or doubled haploid plant of the deposited line may be used in a method for producing a parental line of the progeny line. In one embodiment, the present invention may provide a haploid plant and/or doubled haploid plant, a plant part of a haploid plant and/or doubled haploid plant, or a seed of a haploid plant and/or doubled haploid plant.

The doubled haploid plant can be produced by doubling chromosomes in a haploid plant or a cell (e.g., Reference Literature 7 below). As a specific example, haploid pollen or a haploid ovule is cultured under predetermined conditions, thereby forming plantlets with In chromosomes. Next, the plantlets are treated with, for example, a chemical substance such as colchicine to double the chromosomes. As a result, the cells of the plantlets have 2n chromosomes (doubled haploids). Then, by growing the thus-treated plantlets, the doubled haploid plants and progeny lines thereof can be obtained.
Reference Literature 7: Jim M. Dunwell, “Haploids in flowering plants: origins and exploitation”, Plant Biotechnology Journal, 2010, volume 8, pp. 377-424

### <Method for Producing Lettuce Plant>

As described above, the method for producing a lettuce plant according to the present invention includes the step of crossing a first lettuce plant with a second lettuce plant, and the first lettuce plant is the lettuce plant according to the present invention. The production method according to the present invention is characterized in that the lettuce plant according to the present invention is used as at least one parent in the crossing step, and there is no particular limitation on other steps and conditions.

The lettuce plant production method of the present invention includes the step of selfing (self-pollinating) the lettuce plant of the present invention. The production method according to the present invention is characterized in selfing the lettuce plant of the present invention, and there is no particular limitation on other steps and conditions.

According to the production method of the present invention, a progeny line of the deposited line can be produced. Regarding the production method of the present invention, reference can be made to the above description on the lettuce plant of the present invention.

In the present invention, the crossing between the first lettuce plant (first parental line) and the second lettuce plant (second parental line) may be, for example, crossing in the same individual (regular self-pollination), crossing between the same clonal individuals or between individuals of a line maintained in an inbred line (quasi-self-pollination), or crossing between different individuals (cross-pollination). In the case of the regular self-pollination, one of the first and second parental lines is a female organ of the same individual, and the other one of the first and second parental lines is pollen of the same individual, for example.

Seeds produced by crossing between the different individuals (cross-pollination) and plants obtained by growing the seeds include first-generation hybrids F1 (hybrid first-generation lines, F1 hybrids).

In the present invention, the first parental line is the lettuce plant of the present invention, which is, for example, the above-described lettuce plant deposited under Accession No. FERM BP-22497 or a progeny line thereof.

There is no particular limitation on the second parental line, and any lettuce plant can be used. The second parental line may be, for example, a lettuce plant of a species that is taxonomically the same as or different from the first parental line. The second parental line may be, for example, the deposited line or a progeny line thereof, or may be any other lettuce plant.

The production method of the present invention may further include, after the crossing step, the step of growing a progeny line obtained in the crossing step, for example. Conditions for growing the progeny line in the growing step may be, for example, conditions commonly used for growing lettuce plants.

The lettuce plant of the present invention can be obtained by, for example, the production method of the present invention.

### <Method for Producing Seeds of Lettuce Plant>

The present invention provides a method for producing lettuce seeds. The lettuce seed production method according to the present invention includes the steps of: selfing a lettuce plant of the deposited line or crossing the lettuce plant of the deposited line with another lettuce plant; and optionally collecting (gathering or harvesting) the resulting seeds. The seed production method of the present invention may provide a plant, a plant part, or a seed by growing a seed of a lettuce plant.

The seed production method of the present invention may be a method for producing a seed derived from the deposited line. In this case, the seed production method of the present invention may include the step of: (a) crossing a plant of the deposited line with another lettuce plant to produce a seed. The seed production method of the present invention may further include the steps of: (b) cultivating a lettuce plant from the seed obtained in the step (a) to produce a lettuce plant derived from the deposited line; and (c) selfing the lettuce plant obtained in the step (b) or crossing the lettuce plant obtained in the step (b) with another lettuce plant to produce an additional lettuce plant derived from the deposited line. The seed production method of the present invention may further include the step of: (d) optionally repeating the steps (b) and (c) one or more times to further produce a lettuce plant(s) derived from the deposited line. In this case, a lettuce plant to be used in a repeated step (b) as the lettuce plant cultivated from the seed obtained in the step (a) may be an additional lettuce plant obtained in the preceding step (c). The “one or more times” refer to, for example, one to ten times, three to seven times, or three to five times. The seed production method of the present invention may further include the step of gathering or harvesting seeds. The seed production method of the present invention may provide a seed produced by the above-described method and a plant or a part of a plant individual, each obtained by growing the seed.

### <Method for Producing Hybrid Lettuce Plant>

The present invention provides a method for producing a hybrid (crossbred) lettuce plant. The hybrid plant production method of the present invention includes the step of crossing the lettuce plant of the present invention with another lettuce plant. The hybrid plant production method of the present invention may further include the step of gathering or harvesting seeds obtained by the crossing. The hybrid plant production method of the present invention may provide a seed produced by the above-described method and a hybrid plant or a part of a hybrid plant individual, each obtained by growing the seed.

### <Method for Introducing New Characteristic>

The present invention provides a method for introducing at least one new trait or characteristic (collectively referred to as “characteristic” hereinafter) into the deposited line. The characteristic introduction method of the present invention can also be referred to as, for example, a method for producing a lettuce plant into which a new characteristic(s) has been introduced. The characteristic introduction method of the present invention includes, for example, the steps of: (a) crossing a plant of a deposited line with a lettuce plant including at least one new characteristic in order to produce one or more progeny lines; and (b) selecting a progeny line including the at least one new characteristic. The characteristic introduction method of the present invention includes, for example, the steps of: (c) crossing the progeny line with the deposited line to produce one or more backcross progeny seeds; and (d) selecting a backcross progeny including the at least one new characteristic and essentially all physiological and morphological characteristics of the deposited line. In the steps (b) and (d), selection of the progeny line having a new characteristic may be performed by, for example, detecting the characteristic or detecting a gene or a molecular marker associated (linked) with the characteristic. Examples of the new characteristic include resistance to Bremia Lactucae (B1: 16 to 37, Bl: 5US to 11US), tolerance to bacterial rot, tolerance to corky root, tolerance to lettuce mosaic virus, resistance to lettuce aphids (Nasonovia ribisnigri), resistant to pea leaf miners (Liriomyza huidobrensis), and resistance to browning at cut surfaces. Introduction of a new characteristic according to the above-described method may be defined as a single-locus conversion.

The characteristic introduction method of the present invention may further include the step of: (e) optionally repeating the steps (c) and (d) one or more times to produce a lettuce plant(s) including the at least one new characteristic. The above-described lettuce plant including a new characteristic(s) may be defined as a lettuce plant including at least one single-locus conversion. In this case, in the characteristic introduction method of the present invention, a progeny line to be used in a repeated step (c) may be a backcross progeny selected in the preceding step (d). A lettuce plant obtained or obtainable in the step (e) may have essentially all physiological and morphological characteristics of the deposited line. Regarding the “essentially all physiological and morphological characteristics”, reference can be made to the above description on the progeny line, in which the term “progeny line” should be considered to be replaced with the term “lettuce plant obtained or obtainable in the step (e)”. The “one or more times” refer to, for example, one to ten times, three to seven times, or three to five times. The characteristic introduction method of the present invention may include the step of gathering or harvesting seeds. The characteristic introduction method of the present invention may provide a seed produced by the above-described method and a plant or a part of a plant individual, each obtained by growing the seed.

### <Method for Introducing Transgene>

The present invention provides a method for producing a plant that is derived from a deposited line and includes at least one new trait or characteristic. The transgene introduction method of the present invention can also be referred to as, for example, a method for producing a lettuce plant into which a new characteristic(s) has been introduced.

The transgene introduction method of the present invention includes, for example, the step of introducing mutation or a transgene that confers at least one new characteristic into a plant of the deposited line. The introduction of mutation or a transgene can be performed, for example, in the same manner as the above-described introduction of mutation or a transgene into a progeny line. The lettuce plant obtained or obtainable by the above-described introduction step may have essentially all physiological and morphological characteristics of the deposited line. Regarding the “essentially all physiological and morphological characteristics”, reference can be made to the above description on the progeny line, in which the term “progeny line” should be considered to be replaced with the term “lettuce plant obtained or obtainable by the introduction step”. The transgene introduction method of the present invention may include the step of gathering or harvesting seeds. The transgene introduction method of the present invention may provide a seed produced by the above-described method and a plant or a part of a plant individual, each obtained by growing the seed. Examples of the new characteristic include resistance to Bremia Lactucae (B1: 16 to 37, B1: 5US to 11US), tolerance to bacterial rot, tolerance to corky root, tolerance to lettuce mosaic virus, resistance to lettuce aphids (Nasonovia ribisnigri), resistant to pea leaf miners (Liriomyza huidobrensis), and resistance to browning at cut surfaces.

### <Lettuce Plant Regenerated Product and Regeneration Method>

The present invention provides a lettuce plant regenerated from a cell culture, a tissue culture, or a protoplast of the deposited line (the regenerated lettuce plant is referred to as “regenerated product” hereinafter). The present invention may provide a cell culture or tissue culture of regenerable cells, or a protoplast derived from a lettuce plant of the deposited line. The above-described cells, tissue, or protoplast may be derived from tissue including a leaf, pollen, an embryo, a cotyledon, a hypocotyl, meristematic cells, a root, a root tip, an anther, a flower, a seed, or a trunk.

The present invention provides a method of growth or propagation of a lettuce plant of the deposited line. The propagation of the lettuce plant of the deposited line may be vegetative propagation of the lettuce plant of the deposited line. In this case, the lettuce plant regeneration method according to the present invention includes, for example, the steps of: (a) collecting propagatable tissue from a plant of the deposited line; (b) culturing the tissue to obtain a grown shoot; and (c) rooting the grown shoot to obtain a rooted plantlet. The lettuce plant regeneration method of the present invention may further include the step of: (d) optionally growing a plant from the rooted plantlet. Regarding a method for effecting the above-described vegetative propagation, reference can be made to Reference Literatures 8 and 9 below, for example. The regeneration method of the present invention may provide, for example, a plantlet, a plant, or a part of a plant individual, each regenerated (produced) by the above-described method. The plant may have essentially all physiological and morphological characteristics of the deposited line. Regarding the “essentially all physiological and morphological characteristics”, reference can be made to the above description on the progeny line, in which the term “progeny line” should be considered to be replaced with the term “regenerated plant”.
Reference Literature 8: Habtamu Gudisa Megersa, “Propagation Methods of Selected Horticultural Crops by Specialized Organs: Review”, Journal of Horticulture, 2017, Volume 4, Issue 2, 1000198
Reference Literature 9: Nitish Kumar et al., “In vitro Plant Propagation: A Review”, Journal of Forest Science, 2011, Vol. 21, No. 2, pp. 61-72

### <Harvest and Processed Product of Lettuce Plant>

The present invention provides a harvest and/or processed product of the deposited line or a progeny line thereof. The harvest is a whole plant or a part of a plant individual, and preferably includes: leaves (e.g., heading leaves and expanded leaves); leaves and roots; or seeds.

The processed product encompasses any product obtained by treating the deposited line or the progeny line. The treatment is not limited to particular treatments, and examples thereof include cutting, slicing, grinding, pureeing, drying, canning, bottling, washing, packaging, freezing, and/or heating. In the deposited line or the progeny line, a plant or a part of a plant individual to be used in the processed product is a leaf, for example. The processed product may be, for example, a product obtained by washing and packaging the deposited line or the progeny line. The processed product may be contained, for example, in a container of any size or shape. Specific examples of the container include bags, boxes, and cartons.

### <Method for Determining Genotype>

The present invention provides a method for determining or detecting the genotype of the deposited line or a progeny line thereof. The genotype determination method of the present invention includes, for example, the steps of: (a) obtaining a nucleic acid sample from the deposited line or a progeny line thereof; and (b) detecting a genome in the nucleic acid sample. In the step (a), as a method for preparing a nucleic acid sample from the deposited line or the progeny line, any method commonly used for preparing a nucleic acid sample from tissue can be used. In the step (b), for example, a polymorphism(s) and/or an allele(s) in the genome contained in the nucleic acid sample is detected. Detection of the polymorphism(s) and/or allele(s) can be performed using, for example, single nucleotide polymorphism (SNP) genotyping, amplified fragment length polymorphism (AFLP) detection, restriction fragment length polymorphism (RFLP) identification for genomic DNA, sequence-characterized amplified region (SCAR) detection for genomic DNA, cleaved amplified polymorphic sequence (CAPS) detection for genomic DNA, random amplified polymorphic detection (RAPD) for genomic DNA, a polymerase chain reaction (PCR), DNA sequencing, an allele specific oligonucleotide (ASO) probe, or a DNA microarray. Detection of the polymorphism(s) and/or allele(s) may be performed by sequencing the base sequence of the genome, for example. In the step (b), one polymorphism and/or allele or two or more polymorphisms and/or alleles in the genomic DNA may be detected. The genotype determination method of the present invention may include the step of storing the result of detecting the polymorphism(s) and/or allele(s) in a computer-readable medium. The present invention may provide a computer-readable medium provided by such a method.

The genotype determination method of the present invention may be applied to, for example, any lettuce plant (lettuce plant of interest) instead of the deposited line or the progeny line. In this case, the genotype determination method of the present invention may further include, for example, the step of determining whether the lettuce plant of interest is the progeny line on the basis of the result obtained in the step (b). The “determination” can also be referred to as, for example, discrimination, estimation, appraisal, or assessment. The determination can be made on the basis of, for example, the concordance rate between the result obtained in the step (b) and the genotype of the deposited line.

### Examples

The present invention will be described specifically below with reference to examples. It is to be noted, however, that the present invention is by no means limited to embodiments described in the following examples.

### (1) Breeding of Deposited Line

A lettuce line with late-bolting and resistant to Fusarium oxysporum f. sp. Lactucae (Fol) Race 1 was crossed with a lettuce line with excellent head-forming properties (capable of forming a large head) and early-maturing, whereby F1 generation seeds were obtained. In 2017, the F1 generation was cultivated, whereby selfed seeds were obtained. In 2018, 640 plants in F2 generation were cultivated. Out of these plants, 13 individuals that were vigorous, excellent in head-forming properties, late-bolting, resistant to Fusarium oxysporum f. sp. Lactucae (Fol) Race 1, and resistant to Fusarium oxysporum f. sp. Lactucae (Fol) Yuma isolate were selected and their seeds were collected. In 2019, plants in F3 generation were cultivated. Out of these plants, 22 individuals that were vigorous, excellent in head-forming properties, late-bolting, resistant to Fusarium oxysporum f. sp. Lactucae (Fol) Race 1, and resistant to Fusarium oxysporum f. sp. Lactucae (Fol) Yuma isolate were selected and their seeds were collected. After that, selection and seed collection were repeated in the same manner. At the time when the F7 generation was obtained in 2023, it was determined that the desired characteristics were fixed, and the breeding was terminated.

In an agricultural field in Yuma, Arizona in the United States, about 2000 plants in F7 generation in total were cultivated (sowing in September, two types of cultivation) in 2023. It was found that there was no variation in traits within lines, whereby the bred lettuce variety was confirmed to be uniform and stable. Then, seeds of a couple of lines out of the lines in the F7 generation were mixed together, and some of them were deposited under Accession No. FERM BP-22497 under the designation of TEXLE213131.

### (2) Characteristics of Deposited Line

The traits and characteristics of plant individuals of the thus-obtained deposited line were evaluated according to the Test Guidelines for Variety Registration provided by the Ministry of Agriculture, Forestry and Fisheries of Japan (MAFF). The results thereof are shown in Table 2 below.

TABLE 2

| Characteristic | | Method of | | | |
|---|---|---|---|---|---|
| No. | Characteristics | Investigation | Note | Evaluation | TEXLE213131 |
| 1 | Seed: color | Observation | 1 | White | 1 |
| | | | 2 | Yellow | |
| | | | 3 | Brown | |
| | | | 4 | Black | |
| 4 | Plant: | Measurement/ | 1 | Very small | 5 |
| | diameter | Observation | 3 | Small | |
| | (maximum | cm | 5 | Medium | |
| | diameter of | | 7 | Large | |
| | plant) | | 9 | Very large | |
| 5 | Plant: degree | Observation | 1 | Absent or | 3 |
| | of overlapping | | | weak | |
| | of upper part | | 2 | Medium | |
| | of leaves | | 3 | Strong | |
| | (degree of | | | | |
| 8 | Leaf: number | Observation | 1 | Absent or | 1 |
| | of divisions | | | very few | |
| | | | 3 | Few | |
| | | | 5 | Medium | |
| | | | 7 | Many | |
| | | | 9 | Very many | |
| 9 | Leaf: shape | Observation | 1 | Triangular | 4 |
| | (only for | | 2 | Lanceolate | |
| | varieties in | | 3 | Medium | |
| | which the | | | oblate | |
| | number of | | 4 | Narrow | |
| | divisions in | | | oblate | |
| | leaves is | | 5 | Circular | |
| | absent or very | | 6 | Broad | |
| | few) | | | elliptic | |
| | | | 7 | Medium | |
| | | | | elliptic | |
| | | | 8 | Narrow | |
| | | | | elliptic | |
| | | | 9 | Linear | |
| | | | 10 | Broad | |
| | | | | obtrullate | |
| | | | 11 | Obovate | |
| | | | 12 | Oblanceolate | |
| 13 | Leaf: | Observation | 1 | Absent or | 1 |
| | anthocyanin | | | very weak | |
| | coloration | | 3 | Weak | |
| | | | 5 | Medium | |
| | | | 7 | Strong | |
| | | | 9 | Very strong | |
| 22 | Leaf: | Observation | 1 | Absent or | 6 |
| | blistering | | | very weak | |
| | | | 3 | Weak | |
| | | | 5 | Medium | |
| | | | 7 | Strong | |
| | | | 9 | Very strong | |
| 24 | Leaf: | Observation | 1 | Absent or | 5 |
| | undulation of | | | very weak | |
| | margin | | 3 | Weak | |
| | | | 5 | Medium | |
| | | | 7 | Strong | |
| | | | 9 | Very strong | |
| 26 | Leaf: depth of | Observation | 1 | Absent or | 5 |
| | incisions of | | | very shallow | |
| | margin | | 3 | Shallow | |
| | | | 5 | Medium | |
| | | | 7 | Deep | |
| | | | 9 | Very deep | |
| 30 | Head: size | Measurement/ | 1 | Very small | 5 |
| | (only for | Observation | 3 | Small | |
| | varieties in | cm² | 5 | Medium | |
| | which the | | 7 | Large | |
| | degree of | | 9 | Very large | |
| | overlapping of | | | | |
| | upper part of | | | | |
| | leaves of plant | | | | |
| | is medium or | | | | |
| | strong) | | | | |
| 31 | Head: shape in | Observation | 1 | Narrow | 4 |
| | longitudinal | | | elliptic | |
| | section | | 2 | Broad | |
| | (only for | | | elliptic | |
| | varieties in | | 3 | Circular | |
| | which the | | 4 | Narrow | |
| | degree of | | | oblate | |
| | overlapping of | | | | |
| | upper part of | | | | |
| | leaves of plant | | | | |
| | is medium or | | | | |
| | strong) | | | | |
| 40 | Time of | Measurement/ | 1 | Very early | 6 |
| | beginning of | Observation | 3 | Early | |
| | bolting | days | 5 | Medium | |
| | (the time at | | 7 | Late | |
| | which 50% of | | 9 | Very late | |
| | test plants | | | | |
| | start to bolt; | | | | |
| | the number of | | | | |
| | days elapsed | | | | |
| | since the | | | | |
| | sowing date) | | | | |
| 59 | Resistance to | Measurement | 1 | Susceptible | 3 |
| | Fusarium | | 2 | Moderately | |
| | oxysporum f. | | | resistant | |
| | sp. Lactucae | | 3 | Highly | |
| | (Fol) Race 1 | | | resistant | |

The lettuce plant of the deposited line has resistant to Fusarium oxysporum f. sp. Lactucae (Fol) Yuma isolate in addition to the characteristics and phenotypes in Table 2 above.

While the present invention has been described above with reference to the exemplary embodiments and examples, the present invention is by no means limited thereto. Various changes and modifications that may become apparent to those skilled in the art may be made in the configuration and specifics of the present invention without departing from the scope of the present invention.

As specifically described above, the present invention can provide a novel lettuce plant. Therefore, the present invention is very useful in the field of agriculture such as breeding, for example.

### <Supplementary Notes>

The whole or part of the exemplary embodiments and examples disclosed above can be described as, but not limited to, the following Supplementary Notes.

### (Supplementary Note 1)

A lettuce plant including:
a lettuce plant identified by Accession No. FERM BP-22497 or a progeny line thereof.

### (Supplementary Note 2)

The lettuce plant according to Supplementary Note 1, wherein
the progeny line includes at least 50% of alleles of the lettuce plant identified by Accession No. FERM BP-22497.

### (Supplementary Note 3)

The lettuce plant according to Supplementary Note 1, wherein
the progeny line includes at least 50% of alleles of the lettuce plant identified by Accession No. FERM BP-22497, and
the progeny line has the following characteristics (1), (2) and (14):
   (1) time of beginning of bolting is “slightly late”;
   (2) resistance to Fusarium oxysporum f. sp. Lactucae (Fol) Race 1 is “highly resistant”; and.
   (14) resistance to Fusarium oxysporum f. sp. Lactucae (Fol) Yuma isolate is “resistant”.

### (Supplementary Note 4)

The lettuce plant according to Supplementary Note 1, wherein
the progeny line includes at least 50% of alleles of the lettuce plant identified by Accession No. FERM BP-22497, and
the progeny line has the following characteristics (1) to (14):
   (1) time of beginning of bolting is “slightly late”;
   (2) resistance to Fusarium oxysporum f. sp. Lactucae (Fol) Race 1 is “highly resistant”;
   (3) the seed color is “white”;
   (4) the plant diameter is “medium”;
   (5) the degree of overlapping of upper part of leaves of a plant is “strong”;
   (6) the number of divisions of a leaf is “absent or very few”;
   (7) the leaf shape is “narrow oblate”;
   (8) anthocyanin coloration of a leaf is “absent or very weak”;
   (9) leaf blistering is “slightly strong”;
   (10) undulation of the margin of a leaf is “medium”;
   (11) the depth of incisions of the margin of a leaf is “medium”;
   (12) the size of a head is “medium”;
   (13) the shape in a longitudinal section of a leaf is “medium oblate”; and
   (14) resistant to Fusarium oxysporum f. sp. Lactucae (Fol) Yuma isolate is “resistant”

### (Supplementary Note 5)

The lettuce plant according to any one of Supplementary Notes 1 to 4, wherein
the lettuce plant is a plant body or a part thereof.

### (Supplementary Note 6)

The lettuce plant according to any one of Supplementary Notes 1 to 5, wherein
the lettuce plant is a seed.

### (Supplementary Note 7)

A method for producing a lettuce plant, including the step of:
crossing a first lettuce plant with a second lettuce plant,
wherein the first lettuce plant is the lettuce plant according to any one of Supplementary Notes 1 to 6.

### (Supplementary Note 8)

A seed of a lettuce line TEXLE213131, a representative sample of which is a lettuce plant seed deposited under Accession No. FERM BP-22497.

### (Supplementary Note 9)

A lettuce plant of a lettuce line TEXLE213131, a representative sample of which is a lettuce plant seed deposited under Accession No. FERM BP-22497.

### (Supplementary Note 10)

A lettuce plant or a part thereof, having essentially all physiological and morphological characteristics of the lettuce plant according to Supplementary Note 9.

### (Supplementary Note 11)

A progeny lettuce plant of the lettuce plant according to Supplementary Note 9, wherein
the progeny lettuce plant includes at least 50% of alleles of the lettuce plant according to Supplementary Note 9, and
the progeny lettuce plant has the following characteristics (1) to (14):
   (1) time of beginning of bolting is “slightly late”;
   (2) resistance to Fusarium oxysporum f. sp. Lactucae (Fol) Race 1 is “highly resistant”;
   (3) the seed color is “white”;
   (4) the plant diameter is “medium”;
   (5) the degree of overlapping of upper part of leaves of a plant is “strong”;
   (6) the number of divisions of a leaf is “absent or very few”;
   (7) the leaf shape is “narrow oblate”;
   (8) anthocyanin coloration of a leaf is “absent or very weak”;
   (9) leaf blistering is “slightly strong”;
   (10) undulation of the margin of a leaf is “medium”;
   (11) the depth of incisions of the margin of a leaf is “medium”;
   (12) the size of a head is “medium”;
   (13) the shape in a longitudinal section of a leaf is “medium oblate”; and
   (14) resistant to Fusarium oxysporum f. sp. Lactucae (Fol) Yuma isolate is “resistant”

### (Supplementary Note 12)

A seed that produces the lettuce plant according to Supplementary Note 11.

### (Supplementary Note 13)

A part of the lettuce plant according to Supplementary Note 9.

### (Supplementary Note 14)

The plant part according to Supplementary Note 13, wherein
the plant part includes a microspore, pollen, an ovary, an ovule, an embryonic sac, an egg cell, a cutting, a root, a trunk, a leaf, a cell, or a protoplast.

### (Supplementary Note 15)

A method for producing a lettuce seed, including:
selfing the lettuce plant according to Supplementary Note 9 or crossing the lettuce plant according to Supplementary Note 9 with another lettuce plant; and
obtaining (collecting) a resulting seed.

### (Supplementary Note 16)

A lettuce seed derived from a lettuce plant, wherein
the lettuce seed is produced by the method according to Supplementary Note 15.

### (Supplementary Note 17)

A lettuce plant or a part thereof produced by growing the lettuce seed according to Supplementary Note 16.

### (Supplementary Note 18)

The lettuce plant or the part thereof according to Supplementary Note 17, wherein
the lettuce plant or the part thereof includes at least 50% of alleles of a lettuce line TEXLE213131, a representative sample of which is a lettuce plant seed deposited under Accession No. FERM BP-22497, and
the lettuce plant or the part thereof has the following characteristics (1) to (14):
   (1) time of beginning of bolting is “slightly late”;
   (2) resistance to Fusarium oxysporum f. sp. Lactucae (Fol) Race 1 is “highly resistant”;
   (3) the seed color is “white”;
   (4) the plant diameter is “medium”;
   (5) the degree of overlapping of upper part of leaves of a plant is “strong”;
   (6) the number of divisions of a leaf is “absent or very few”;
   (7) the leaf shape is “narrow oblate”;
   (8) anthocyanin coloration of a leaf is “absent or very weak”;
   (9) leaf blistering is “slightly strong”;
   (10) undulation of the margin of a leaf is “medium”;
   (11) the depth of incisions of the margin of a leaf is “medium”;
   (12) the size of a head is “medium”;
   (13) the shape in a longitudinal section of a leaf is “medium oblate”; and
   (14) resistant to Fusarium oxysporum f. sp. Lactucae (Fol) Yuma isolate is “resistant”

### (Supplementary Note 19)

The lettuce plant or the part thereof according to Supplementary Note 17, wherein
the lettuce plant or the part thereof has essentially all physiological and morphological characteristics of a lettuce line TEXLE213131, a representative sample of which is a lettuce plant seed deposited under Accession No. FERM BP-22497.

### (Supplementary Note 20)

The lettuce plant or the part thereof according to Supplementary Note 18, wherein
at least one characteristic of the lettuce plant or the part thereof is modified.

### (Supplementary Note 21)

The lettuce plant or the part thereof according to Supplementary Note 20, wherein
the at least one characteristic is modified by causing mutagenesis.

### (Supplementary Note 22)

A method for producing a seed of a lettuce plant derived from the lettuce plant according to Supplementary Note 9, the method including:
(a) crossing a lettuce line TEXLE213131, a representative sample of which is a lettuce plant seed deposited under Accession No. FERM BP-22497, with another lettuce plant to produce a seed;
(b) growing a lettuce plant from the seed obtained in the step (a) to produce a lettuce plant derived from the lettuce line TEXLE213131;
(c) selfing the lettuce plant obtained in the step (b) or crossing the lettuce plant obtained in the step (b) with another lettuce plant to produce an additional lettuce plant derived from the lettuce line TEXLE213131; and
(d) optionally repeating the steps (b) and (c) one or more times to further produce a lettuce plant derived from the lettuce line TEXLE213131, wherein a lettuce plant used in a repeated step (b) is grown from an additional lettuce plant obtained in a preceding step (c).

### (Supplementary Note 23)

A seed produced by the method according to Supplementary Note 22, wherein
the seed includes at least 50% of alleles of the lettuce plant according to Supplementary Note 9, and
a lettuce plant grown from the seed has the following characteristics (1) to (14):
   (1) time of beginning of bolting is “slightly late”;
   (2) resistance to Fusarium oxysporum f. sp. Lactucae (Fol) Race 1 is “highly resistant”;
   (3) the seed color is “white”;
   (4) the plant diameter is “medium”;
   (5) the degree of overlapping of upper part of leaves of a plant is “strong”;
   (6) the number of divisions of a leaf is “absent or very few”;
   (7) the leaf shape is “narrow oblate”;
   (8) anthocyanin coloration of a leaf is “absent or very weak”;
   (9) leaf blistering is “slightly strong”;
   (10) undulation of the margin of a leaf is “medium”;
   (11) the depth of incisions of the margin of a leaf is “medium”;
   (12) the size of a head is “medium”;
   (13) the shape in a longitudinal section of a leaf is “medium oblate”; and
   (14) resistant to Fusarium oxysporum f. sp. Lactucae (Fol) Yuma isolate is “resistant”

### (Supplementary Note 24)

A lettuce plant produced by growing the seed according to Supplementary Note 23.

### (Supplementary Note 25)

A method for introducing at least one new characteristic into the lettuce plant according to Supplementary Note 9, the method including:
(a) crossing a lettuce line TEXLE213131, a representative sample of which is a lettuce plant seed deposited under Accession No. FERM BP-22497, with a lettuce plant having at least one new characteristic to produce one or more progenies;
(b) selecting a progeny including the at least one new characteristic;
(c) crossing the selected progeny with the lettuce line TEXLE213131 to produce one or more backcross progenies;
(d) selecting a backcross progeny including the at least one new characteristic and having essentially all physiological and morphological characteristics of the lettuce line TEXLE213131; and
(e) optionally repeating the steps (c) and (d) one or more times to produce a lettuce plant(s) including the at least one new characteristic and having essentially all physiological and morphological characteristics of the lettuce line TEXLE213131, wherein a lettuce plant used in a repeated step (c) is a backcross progeny selected in a preceding step (d).

### (Supplementary Note 26)

A lettuce plant produced by the method according to Supplementary Note 25.

### (Supplementary Note 27)

A method for producing a lettuce plant that is derived from a lettuce line TEXLE213131 and includes at least one new characteristic, the method including:
introducing mutation or a transgene that confers at least one new characteristic into a lettuce line TEXLE213131, a representative sample of which is a lettuce plant seed deposited under Accession No. FERM BP-22497.

### (Supplementary Note 28)

A lettuce plant produced by the method according to Supplementary Note 27.

### (Supplementary Note 29)

A method for producing one or more lettuce heading leaves as a food, including the step of:
harvesting one or more heading leaves of the lettuce plant according to Supplementary Note 9 or the one or more heading leaves, one or more expanded leaves, and a stem of the lettuce plant according to Supplementary Note 9.

### (Supplementary Note 30)

A processed product of the lettuce plant according to Supplementary Note 9, including:
a cut, sliced, ground, pureed, dried, canned, bottled, washed, packaged, frozen and/or heat-treated heading leaf.

### (Supplementary Note 31)

A method for determining a genotype of the lettuce plant according to Supplementary Note 9 or a progeny line thereof, the method including:
(a) obtaining a nucleic acid sample from the lettuce plant according to Supplementary Note 9; and
(b) detecting a polymorphism in the nucleic acid sample.

### (Supplementary Note 32)

A tissue culture of regenerable cells or regenerable protoplasts derived from the lettuce plant according to Supplementary Note 9 or a progeny line thereof.

### (Supplementary Note 33)

The tissue culture according to Supplementary Note 32, wherein
the cells or the protoplasts are derived from a leaf, pollen, an embryo, a cotyledon, a hypocotyl, a meristematic cell, a root, a root tip, an anther, a flower, a seed, or a stem.

### (Supplementary Note 34)

A lettuce plant regenerated from the tissue culture according to Supplementary Note 33.

### (Supplementary Note 35)

The lettuce plant according to Supplementary Note 34, wherein
the lettuce plant has essentially all physiological and morphological characteristics of a lettuce line TEXLE213131, a representative sample of which is a lettuce plant seed deposited under Accession No. FERM BP-22497.

### (Supplementary Note 36)

A method for vegetative propagation of the lettuce plant according to Supplementary Note 9, the method including:
(a) collecting propagatable tissue from a lettuce plant of a lettuce line TEXLE213131, a representative sample of which is a lettuce plant seed deposited under Accession No. FERM BP-22497;
(b) culturing the tissue to obtain a grown shoot;
(c) rooting the grown shoot to obtain a rooted plantlet; and
(d) optionally growing the rooted plantlet into a plant.

### (Supplementary Note 37)

A lettuce plantlet or lettuce plant produced by the method according to Supplementary Note 36, wherein
the lettuce plantlet or lettuce plant has essentially all physiological and morphological characteristics of the lettuce line TEXLE213131, a representative sample of which is a lettuce plant seed deposited under Accession No. FERM BP-22497.

### (Supplementary Note 38)

A lettuce plant including:
a lettuce plant identified by Accession No. FERM BP-22497.

### (Supplementary Note 39)

A lettuce plant including:
a progeny line of the lettuce plant according to Supplementary Note 38,
wherein the progeny line has the following characteristics (1) to (14):
   (1) time of beginning of bolting is “slightly late”;
   (2) resistance to Fusarium oxysporum f. sp. Lactucae (Fol) Race 1 is “highly resistant”;
   (3) the seed color is “white”;
   (4) the plant diameter is “medium”;
   (5) the degree of overlapping of upper part of leaves of a plant is “strong”;
   (6) the number of divisions of a leaf is “absent or very few”;
   (7) the leaf shape is “narrow oblate”;
   (8) anthocyanin coloration of a leaf is “absent or very weak”;
   (9) leaf blistering is “slightly strong”;
   (10) undulation of the margin of a leaf is “medium”;
   (11) the depth of incisions of the margin of a leaf is “medium”;
   (12) the size of a head is “medium”;
   (13) the shape in a longitudinal section of a leaf is “medium oblate”; and
   (14) resistant to Fusarium oxysporum f. sp. Lactucae (Fol) Yuma isolate is “resistant”

### (Supplementary Note 40)

A lettuce plant including:
a hybrid first-generation line of the lettuce plant according to Supplementary Note 38 or 39.

### (Supplementary Note 41)

The lettuce plant according to any one of Supplementary Notes 38 to 40, wherein
the lettuce plant is a plant body or a part thereof.

### (Supplementary Note 42)

The lettuce plant according to any one of Supplementary Notes 38 to 41, wherein
the lettuce plant is a seed.

### (Supplementary Note 43)

A method for producing a lettuce plant, including the step of:
selfing the lettuce plant according to any one of Supplementary Notes 38 to 42.

### (Supplementary Note 44)

A method for producing a lettuce plant, including the step of:
crossing the lettuce plant according to any one of Supplementary Notes 38 to 42 with another lettuce plant.

### (Supplementary Note 45)

The production method according to Supplementary Note 43 or 44, further including the step of:
collecting a seed.

### [FIG. 1A]

Characteristic No. 5 Plant: degree of overlapping of upper part of leaves
Note: 1 absent or weak
Note: 2 medium
Note: 3 strong

### [FIG. 1B]

Characteristic No. 8 Leaf: number of divisions
Note: 1 absent or very few
Note: 3 few
Note: 5 medium
Note: 7 many
Note: 9 very many

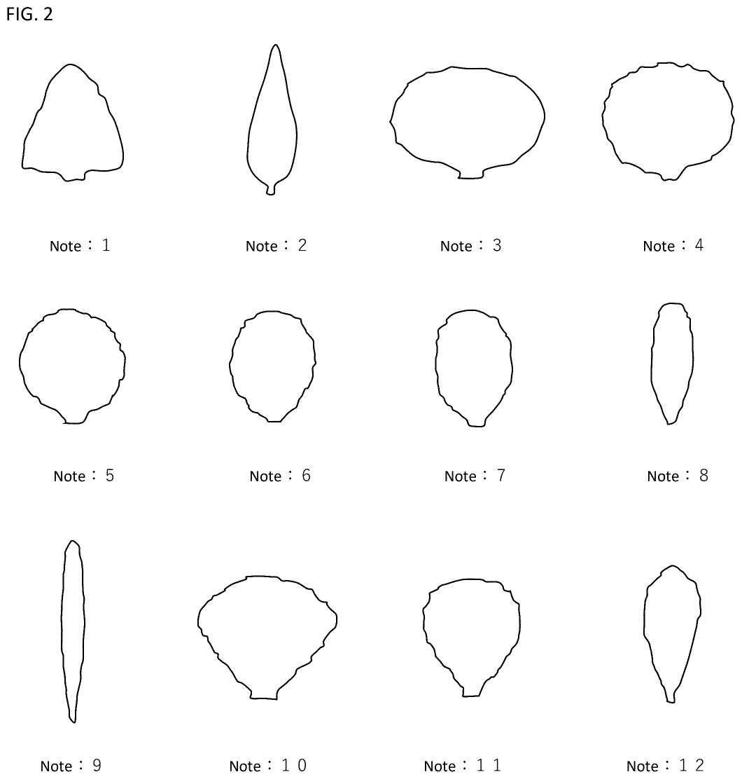

### [FIG. 2]

Characteristic No. 9 Leaf: shape
Note: 1 triangular
Note: 2 lanceolate
Note: 3 medium oblate
Note: 4 narrow oblate
Note: 5 circular
Note: 6 broad elliptic
Note: 7 medium elliptic
Note: 8 narrow elliptic
Note: 9 linear
Note: 10 broad obtrullate
Note: 11 obovate
Note: 12 oblanceolate

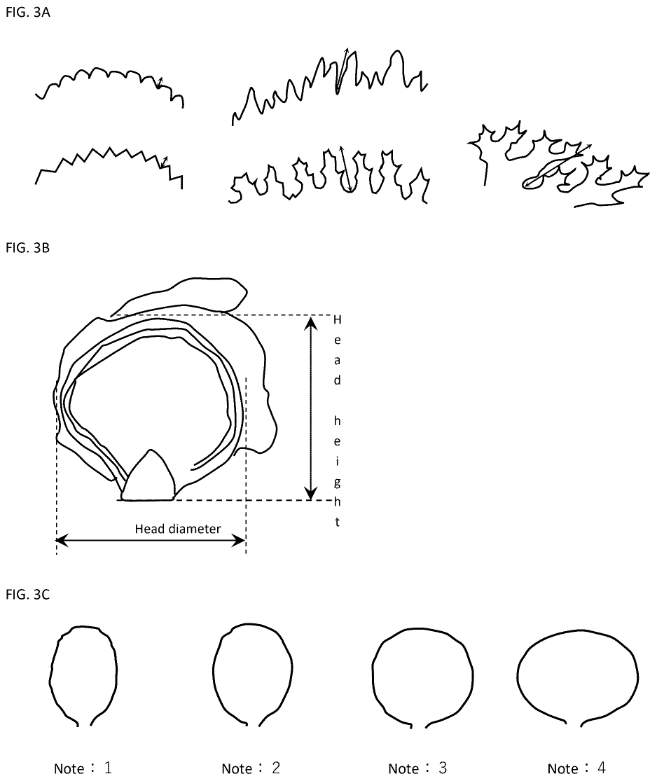

### [FIG. 3A]

Characteristic No. 26 Leaf: depth of incisions of margin

### [FIG. 3B]

Characteristic No. 30 Head: size

### [FIG. 3C]

Characteristic No. 31 Head: shape in longitudinal section
Note: 1 narrow elliptic
Note: 2 broad elliptic
Note: 3 circular
Note: 4 medium oblate

1. A lettuce seed of a lettuce line TEXLE213131, a representative sample of the seed being deposited under Accession No. FERM BP-22497.

2. A lettuce plant grown from the seed according to claim 1.

3. A lettuce plant having essentially all physiological and morphological characteristics of the lettuce plant according to claim 2.

4. A tissue culture of the lettuce plant according to claim 2.

5. A lettuce plant regenerated from the tissue culture according to claim 4.

6. A lettuce plant of a lettuce line TEXLE213131, wherein
a representative sample of a seed of the plant is deposited under Accession No. FERM BP-22497, and the plant further includes mutation and/or a transgene.

7. A lettuce plant of a lettuce line TEXLE213131, wherein
a representative sample of a seed of the plant is deposited under Accession No. FERM BP-22497, and the plant further includes a single-locus conversion.

8. A progeny line of the lettuce plant according to claim 2, wherein
the progeny line includes at least 50% of alleles of a lettuce line TEXLE213131 and has the following characteristics (1), (2), and (14):
(1) time of beginning of bolting is “slightly late”;
(2) resistance to Fusarium oxysporum f. sp. Lactucae (Fol) Race 1 is “highly resistant”; and.
(14) resistance to Fusarium oxysporum f. sp. Lactucae (Fol) Yuma isolate is “resistant”.

9. A part of the lettuce plant according to claim 2.

10. A lettuce plant of a hybrid first-generation line or a part thereof, wherein
a first parental line is the lettuce plant according to claim 2 and a second parental line is another lettuce plant.

11. A method for producing a lettuce plant, comprising the step of:
selfing the lettuce plant according to claim 2.

12. A method for producing a lettuce plant, comprising the step of:
crossing the lettuce plant according to claim 2 with another lettuce plant.

13. A method for producing a lettuce plant, comprising:
introducing mutation to the lettuce seed according to claim 1; and
growing the seed.

14. A method for producing a lettuce plant, comprising:
introducing mutation to the lettuce plant according to claim 2.
